(19) United States
(12) Patent Application Publication
(10) Pub. No.: US 2026/0021442 A1
(43) Pub. Date: Jan. 22, 2026

(54) Oxygen Concentrator Sorbent Bed Equalization and Feed Manifolds and Method of Using the Same

(71) Applicant: TDA Research, Inc., Wheat Ridge, CO (US)

(72) Inventors: Gökhan Alptekin, Boulder, CO (US); Ambalavanan Jayaraman, Highlands Ranch, CO (US); Brandon Gushlaw, Arvada, CO (US); Douwe Bruinsma, Louisville, CO (US)

(21) Appl. No.: 18/779,860

(22) Filed: Jul. 22, 2024

(51) Int. Cl. B01D 53/047; A61M 16/10; B01D 53/04

(52) U.S. Cl. CPC B01D 53/0476; A61M 16/101; B01D 53/0462; B01D 2256/12; B01D 2259/40013; B01D 2259/40028; B01D 2259/40035; B01D 2259/402; B01D 2259/403; B01D 2259/4533; B01D 2259/4541; B01D 2259/455

(57) ABSTRACT

An oxygen concentrator device comprising at least 2 separation vessels with a feed end and a product end, and a set of manifolds comprising at least one feed manifold attached to the feed end of the separation vessels and at least one equalization manifold attached to the product end of the separation vessels. The feed manifold comprises compression ports and valving; vacuum ports and valving; and, an upstream headspace equaling 2-6% of the total sorbent volume in the combined sieve beds. The equalization manifold comprises equalization ports and valving; and, a downstream headspace equaling 2-6% of the total sorbent volume in the combined sieve beds. The ratio of compression, vacuum, and equalization Cv to total sorbent volume is

$$0.0005 - 0.005 ( \frac{gal}{min} ) ( \frac{1}{{L}_{S}} ) .$$

Also, a method for concentrating oxygen, providing the set of manifolds described above and operating the oxygen concentrator without separately purging the sieve bed with an unused gas.

### STATEMENT OF GOVERNMENT INTEREST

This invention was made in part using U.S. government funding through the United States Marine Corps, SBIR Phase II Contract No. M67854-16-C-6504 and SBIR Phase 11 Contract No. M67854-19-C-5139. The government has certain rights in the invention.

### FIELD OF THE INVENTION

The present invention relates generally to air separation devices for portable oxygen concentrators and, in particular for ambulatory and stationary patient use of medical grade oxygen delivery as supplemental care.

### BACKGROUND OF THE INVENTION

Portable oxygen concentrator modalities mainly exist in one of three forms: high pressure cylinders, cryogenic liquid cylinders, and pressure-swing-adsorption (PSA)/vacuum-pressure-swing-adsorption (VPSA) using air separation adsorbents. Traditional portable oxygen concentrators operate under PSA/VPSA using air separation adsorbents specifically for producing USP grade oxygen. Existing portable oxygen concentrators (POCs) are not battery operated, are high powered, and/or weigh over 32 lbs., making transportation for ambulatory patients difficult.

PSA gas separation systems operate by preferentially adsorbing gases. Gas that is less preferentially adsorbed passes through an adsorbent bed and exits the bed on the product side at a higher concentration than at the inlet. The product side of the bed is opposite the feed side. At some state during the separation process, the flow into the sorbent bed is stopped and the gas that is preferentially adsorbed by the sorbent is removed by depressurizing the sorbent bed from the feed side. The depressurization can be either to ambient pressure conditions, or to sub-ambient pressure conditions. Additionally, the sorbent bed may be purged with a gas that is not readily adsorbed by the sorbent. When the sorbent bed is cycled in this manner, the product end of the bed is not exposed to high concentrations of the gases that are more preferentially adsorbed by the sorbent.

Fundamentally, PSA or VPSA operation consists of four states: Feed State, Purge State, Regeneration State (Ambient or Vacuum), and Equalization State. In general, each of these four states require numerous valves, manifolds, and pneumatic lines. Current designs for POCs lack the ability to continuously provide high-grade oxygen without a distinct Purge State (and thus additional weight, valving, pneumatic lines).

Each current concentrator using PSA or VPSA cycle operation has a set of unique features and variables for maximizing the performance of any one specific design. For instance, some devices use lower pressure swing adsorption to design smaller and lighter oxygen concentrators which have material properties that can minimize weight to yield modulus ratios at the lower pressure requirements. U.S. Pat. Nos. 5,246,676 and 5,393,326 reduce throttling losses to improve the efficiency of the system. Some, such as the PSA devices disclosed in U.S. Pat. Nos. 3,176,446, 6,565,635B2, 5,133,784, use varying rotating or reciprocating mechanisms for cycling the system through pressure and vacuum states. U.S. Pat. No. 6,063,161 rotates assemblies with valving functionalities connected to the adsorbent beds while U.S. Pat. No. 6,451,095 rotates the adsorbent beds around the manifolds for similar operation, but a unique design.

There is a need for a system or manifold for PSA or VPSA operation which relies on fewer cycle states, to reduce the corresponding hardware requirements such that the PSA or VPSA system is more compact and portable. There is also a need for an improved system or manifold that can be adapted to gas separators of varying sizes, while maximizing both throughput and oxygen performance. There is a need for a manifold system that eliminates the Purge state, reduces hardware requirements, and separation system size, mass, or cube. There is a need for a continuous flow oxygen delivery system that is truly portable, so transport and battery use for applicable time in ambulatory or stationary use-cases for oxygen therapy are feasible.

### SUMMARY OF THE INVENTION

The present disclosure relates to an oxygen concentrator with a set of manifolds that overcomes the limitations of the prior art and eliminates the need for a purge mechanism in a PSA or VPSA concentrator. The present disclosure also relates to a method of operating a PSA/VPSA oxygen concentrator without a separate purge step (purge state) and still allowing an equalization state which conserves energy from the compressed gas stream for producing oxygen. More so, the manifolds allow for the use of pneumatic valving that inherently possesses higher valve flow coefficients for allowing feed and equalization gas flow rates that make the rapid cycling PSA/VPSA highly efficient compared to other portable oxygen concentrators. The cycling time is also reduced by the use of the adsorbent bed manifolds which have radial seals meaning headspace is completely eliminated between the sieve material and manifolds (inlet and outlet). The rapid cycling to the desired adsorption pressures leads to a larger volume of air per unit time introducing more oxygen into the delivery system that can be extended to any compressor and vacuum for improving the drive flow. The manifolds described herein offer a design which can be scaled for a wide range of portable oxygen concentrator output flow rates consisting of USP grade oxygen. The major advantages of this design in the manifolds are: reducing the need of a purge mechanism, reducing the mass or volume of the overall system by eliminating the hardware for the purge mechanism, high flow rate, pneumatic actuation of manifold ported valving, and reduced headspace in the adsorbent vessels. Altogether, the disclosed manifolds have applications in portable (lightweight, highly efficient, battery operable, and long lasting) oxygen concentrators and continuous gas separator systems.

The set comprises two manifolds declared as the feed manifold (inlet) and the equalization manifold (outlet). The manifolds manage the gas streams with pneumatic valves defined as the Main Valve (inlet, feed manifold) and the Equalization Valve (outlet, equalization manifold). The manifolds also couple two reactor vessels which are annular tubes used to hold the adsorbent for oxygen-nitrogen separation. Each manifold has a unique set of features for plumbing the pneumatic pressure lines required for valve actuation that allow air flow from the inlet compressor port to the Main Valve and Equalization Valve. The Main Valve allows the Feed and Regeneration states to occur while the Equalization Valve allows for the combined Equalization and Purge states of the PSA/VPSA operation states, eliminating a separate Purge state with a separate input line. The outlet manifold has a unique set of features to allow the use of specific miniature umbrella check valves, so high O₂ product gas that has passed through the adsorbent and been separated from the compressed air can leave the system to another subsystem for patient deliver. Finally, the manifolds both have similar radial rod seal glands for using O-ring seals to effectively hold pressures up to 100 psia and down to 2 psia.

The disclosure provides a pressure-swing-adsorption (PSA) or vacuum-pressure-swing-adsorption (VPSA) oxygen concentrator device comprising: at least 2 sorbent beds, each having a feed end and a product end; at least one feed manifold attached to the feed end of the at least 2 sorbent beds; and at least one equalization manifold attached to the product end of the at least 2 sorbent beds; wherein, the oxygen concentrator device does not comprise a purge mechanism; wherein, the oxygen concentrator device weighs less than 32 pounds; and, wherein, the oxygen concentrator device produces a concentrated gas stream of at least 70-95% oxygen.

The disclosure provides a pressure-swing-adsorption (PSA) or vacuum-pressure-swing-adsorption (VPSA) oxygen concentrator device comprising: at least 2 sorbent beds, each having a feed end, a product end, and a total sorbent volume within the combined sorbent beds; at least one feed manifold attached to the feed end of the at least 2 sorbent beds; and, at least one equalization manifold attached to the product end of the at least 2 sorbent beds. The at least one feed manifold further comprises: at least one compression port connecting the at least 2 sorbent beds to a compression valve, wherein the compression valve has a compression valve flow coefficient (Cv); at least one vacuum port connecting the at least 2 sorbent beds to a vacuum valve, wherein the vacuum valve has a vacuum valve flow coefficient (Cv); and, an upstream headspace, comprising a compression headspace between the sorbent beds and the compression valve, and a vacuum headspace between the sorbent beds and the vacuum valve. The at least one equalization manifold further comprises: at least one equalization port connecting the at least 2 sorbent beds to an equalization valve, wherein the equalization valve has an equalization valve flow coefficient (Cv); and, a downstream headspace, comprising an equalization headspace between the sorbent beds and the equalization valve. The equalization manifold may further comprise at least one check valve between each of the at least 2 sorbent beds and the equalization valve, wherein the check valve connects each of the at least 2 sorbent beds to an O₂ product chamber. A ratio of the compression Cv to the total sorbent volume is

$$0.0005 - 0.005 ( \frac{gal}{min} ) ( \frac{1}{{L}_{s}} ) ;$$

a ratio of the vacuum Cv to the total sorbent volume is

$$0.0005 - 0.005 ( \frac{gal}{min} ) ( \frac{1}{{L}_{s}} ) ;$$

a ratio of the equalization Cv to the total sorbent volume is

$$0.0005 - 0.005 ( \frac{gal}{min} ) ( \frac{1}{{L}_{s}} ) .$$

The upstream headspace is 2-6% of the total sorbent volume, and the downstream headspace is 2-6% of the total sorbent volume.

In a preferred embodiment, the upstream headspace is 3-5% of the total sorbent volume. More preferably, the upstream headspace is 4.25-4.75% of the total sorbent volume. Optionally, the vacuum headspace is 0.5% of the total sorbent volume and the compression headspace is 0.5-2.5% of the total volume. Also optionally, the vacuum headspace is 1-2% of the total sorbent volume and the compression headspace is 0.5-1.5% of the total sorbent volume. Also optionally, the vacuum headspace is 1.2-1.6% of the total sorbent volume and the compression headspace is 0.55-0.70% of the total sorbent volume.

Preferably, the downstream headspace is 3-5% of the total sorbent volume. Optionally, the downstream headspace is 4.25-4.75% of the total sorbent volume.

In an embodiment, the feed manifold comprises a five-port-two-way valve and the equalization manifold comprises a two-port-two-way valve.

In an embodiment, the ratio of the compression Cv to the total sorbent volume, the ratio of the vacuum Cv to the total sorbent volume, and the ratio of the equalization Cv to the total sorbent volume are each

$$0.001 - 0.003 ( \frac{gal}{min} ) ( \frac{1}{{L}_{S}} ) .$$

In another embodiment, the total sorbent volume is 100-6000 mL.

In an optional embodiment, the at least 2 sorbent beds comprise at least 3 sorbent beds. The at least 3 sorbent beds may comprise an odd number of sorbent beds, wherein the odd number of sorbent beds comprises at least 2 sorbent beds operating in parallel. The at least 3 sorbent beds may comprise an even number of sorbent beds, wherein the even number of sorbent beds comprises at least 2 sorbent beds operating in parallel.

Optionally, the oxygen concentrator device further comprises a temperature-swing-adsorption device.

Preferably, the oxygen concentrator device does not comprise a purge mechanism. Also preferably, the oxygen concentrator device weighs less than 32 lbs.

In an embodiment, the at least one feed manifold further comprises a pneumatic port connecting to at least one pressure line for actuation of the compression valve and the vacuum valve.

The disclosure also provides a method of concentrating oxygen for medical purposes, the steps comprising: a) providing a pressure-swing-adsorption (PSA) or vacuum-pressure-swing-adsorption (VPSA) oxygen concentrator device comprising at least 2 sorbent beds having a feed end and a product end, and a total sorbent volume within the combined sorbent beds; b) attaching at least one feed manifold to the feed end of the at least 2 sorbent beds, wherein the at least one feed manifold comprises: at least one compression port connecting the at least 2 sorbent beds to a compression valve; at least one vacuum port connecting the at least 2 sorbent beds to a vacuum valve; and, an upstream headspace; c) attaching at least one equalization manifold to the product end of the at least 2 sorbent beds, wherein the at least one equalization manifold comprises: at least one equalization port connecting the at least 2 sorbent beds to an equalization valve; and, a downstream headspace. The equalization manifold may also comprise an O₂ product chamber connected to each of the at least 2 sorbent beds; d) operating the PSA or VPSA oxygen concentrator device, wherein operating the PSA or VPSA oxygen concentrator device does not comprise separately purging the sieve bed with an unused gas; and, e) producing a concentrated gas stream comprising 50-90% oxygen.

Optionally, the PSA or VPSA oxygen concentrator device further comprises a temperature-swing-adsorption device.

Preferably the upstream headspace is 2-6% of the total sorbent volume. Also preferably, the downstream headspace is 2-6% of the total sorbent volume.

In an embodiment, the compression valve has a compression valve flow coefficient (Cv), and a ratio of the compression Cv to the total sorbent volume is

$$0.0005 - 0.005 ( \frac{gal}{min} ) ( \frac{1}{{L}_{S}} ) .$$

In another embodiment, the vacuum valve has a vacuum valve flow coefficient (Cv), and a ratio of the vacuum Cv to the total sorbent volume is

$$0.0005 - 0.005 ( \frac{gal}{min} ) ( \frac{1}{{L}_{S}} ) .$$

In another embodiment, the equalization valve has an equalization valve flow coefficient (Cv), and a ratio of the equalization Cv to the total sorbent volume is

$$0.0005 - 0.005 ( \frac{gal}{min} ) ( \frac{1}{{L}_{S}} ) .$$

Optionally, the feed manifold further comprises a pneumatic port connecting to at least one pressure line for actuation of the compression valve and the vacuum valve.

Preferably, the concentrated gas stream comprises at least 70-90% oxygen. More preferably, the concentrated gas stream comprises at least 80-90% oxygen.

### BRIEF DESCRIPTION OF DRAWINGS

FIG. 1A: First view of set of manifolds on two-sieve-bed gas separator system with attached valving.

FIG. 1B: Second view of set of manifolds on two-sieve-bed gas separator system with attached valving.

FIG. 2A: Feed manifold top view.

FIG. 2B: Feed manifold inner view.

FIG. 2C: Feed manifold outer side view.

FIG. 3A: Equalization manifold top view.

FIG. 3B: Equalization manifold inner side view.

FIG. 3C: Equalization manifold bottom view.

FIG. 4A: Feed manifold viewing direction for cross-sectional views.

FIG. 4B: Feed manifold with horizontal cross-sectional cut, view from below.

FIG. 4C: Feed manifold with vertical cross-sectional cut, view from outer side.

FIG. 5A: Equalization manifold viewing direction for cross-sectional views.

FIG. 5B: Equalization manifold with vertical cross-sectional cut, view from inner side.

FIG. 5C: Equalization manifold with horizontal cross-sectional cut, view from above.

FIG. 6A: 2D rendition from horizontal cross-sectional cut of feed manifold possible equalization headspace.

FIG. 6B: 3D rendition of volume of feed manifold possible equalization headspace.

FIG. 7A: 2D rendition from vertical cross-sectional cut of feed manifold possible vacuum headspace.

FIG. 7B: 3D rendition of volume of feed manifold possible vacuum headspace.

FIG. 8A: 2D rendition from horizonal cross-sectional cut of equalization manifold possible equalization headspace.

FIG. 8B: 3D rendition of volume of equalization manifold possible equalization headspace.

FIG. 9A: Feed and Equalization Manifolds and sieve beds during a full cycle of O₂ purification, showing compression, vacuum, and equalization headspaces with the first sieve bed in compression state and second sieve bed in vacuum/regeneration state.

FIG. 9B: Feed and Equalization Manifolds and sieve beds during a full cycle of O₂ purification, showing compression, vacuum, and equalization headspaces with both sieve beds in equalization state.

FIG. 9C: Feed and Equalization Manifolds and sieve beds during a full cycle of O₂ purification, showing compression, vacuum, and equalization headspaces with the first sieve bed in vacuum state the second sieve bed in compression state.

FIG. 9D: Feed and Equalization Manifolds and sieve beds during a full cycle of O₂ purification, showing compression, vacuum, and equalization headspaces with both sieve beds in equalization state.

### DETAILED DESCRIPTION OF THE INVENTION

Oxygen concentrator modules vary in design to perform operation states of a traditional pressure-swing-adsorption or vacuum-pressure-swing-adsorption gas separation process. Each PSA/VPSA separator uses an optimized transition of states called Feed or Pressure, Regeneration or Vacuum, Purge, and Equalization—the design disclosed herein eliminates the need for a separate Purge mechanism, and provides compact manifolds for efficient passing through the other three states. Even without the purge mechanism, the oxygen concentrator described herein produces an oxygen stream of at least 50-90% pure oxygen.

Adsorbent vessels, pilot air vessel, buffer tank vessel, and valves are used for the complete operation of the PSA/VPSA system, with the manifolds providing optimized valving. The use of the two manifolds mounts the Main/Feed valving and EQ/Equalization valving, supplies pilot air for pneumatics, and holds packed bed reactors while operating the states of the PSA/VPSA system to allow high O₂ production from air gas separation. The unit has a design with minimal pressure drop for maximum compression and vacuum flow. The design allows for a compact system that can hold the adsorbent vessels, state control valves, pressure sensors, pump drivers, and control board on one compact unit.

In the Summary of the Invention above and the Detailed Description of the Invention, and in the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

Unless defined otherwise, all technical and scientific terms used herein have the same meaning as commonly understood by one of the ordinary skill in the art to which the invention belongs.

“Comprises” and grammatical equivalents thereof are used herein to mean that other components, ingredients, steps, etc. are optionally present. For example, an article ‘comprising’ (or ‘which comprises’) component A, B, and C can consist of (i.e., contain only) components A, B, and C, or can contain not only components A, B, and C but also one or more other components.

The term “at least” followed by a number is used herein to denote the start of a range beginning with that number (which may be a range having an upper limit or no upper limit, depending on the variable being defined). For example, “at least 2” means 2 or more than 2. The term “at most” followed by a number is used herein to denote the end of a range ending with that number (which may be a range having 1 or 0 as its lower limit, or a range having no lower limit, depending on the variable being defined). For example, “at most 4” means 4 or less than 4, and “at most 40%” means 40% or less than 40%. When, in this specification, a range is given as “(a first number) to (a second number)” or “(a first number)−(“a second number)”, this means a range whose lower limit is the first number and whose upper limit is the second number. For example, “2-6%” means a range whose lower limit is 2% and whose upper limit is 6%, where both 2% and 6% are included in that range.

“Set of manifolds” as used in the claims means more than one manifold, with the more than one manifolds working in tandem with one another and/or having qualities (such as headspaces or functionalities) that impact the design of the other manifolds in the set.

“Sorbent” is defined as an insoluble material or mixture of materials capable of selectively adsorbing certain gases from a multicomponent gas stream. The sorbent is present in the sieve bed and may comprise one or more than one sorbent materials mixed together or kept separate, but forming one continuous layer of sorbent. The choice is sorbent is dependent on the input gas and desired concentrated product gas—the sorbent should sufficiently adsorb the non-preferred components of the input gas. Preferably, the sorbent is a molecular sieve. Nonlimiting examples of sorbent materials include zeolites, alumina, activated carbon, silicas, and synthetic resins. “Sorbent bed” and “sieve bed” may be used interchangeably throughout the specification as the location of the sorbent within the adsorption vessels.

“Total sorbent volume” is defined as the combined volume of all sorbent material in all sieve beds in an entire gas separator system. For example, if the gas separator system uses 4 adsorbent vessels with sieve beds, the total sorbent volume is the volume of sorbent in the first sieve bed, plus the volume of sorbent in the second sieve bed, plus the volume of sorbent in the third sieve bed, plus the volume of sorbent in the fourth sieve bed.

“Purge mechanism” is defined as any valving, tubing, channels, hardware, switches, pneumatic lines, stored gas, chambers, manifolds, etc. that is used for enabling a separate Purge state in the PSA or VPSA cycle. Separate Purge state is when an unused, fresh, or external gas is blown across the sorbent bed to effectively reset the sorbent for adsorption in a succeeding Feed/compression state, or when a gas is blown across the sorbent bed while the oxygen concentrator is not actively in a vacuum state or a compression state. The Purge Step is distinct from the Feed, Regeneration, and Equalization steps. The purge mechanism includes any element on the oxygen concentrator which facilitates a separate Purge state, and does not facilitate any of the Feed, Regenerator, or Equalization states.

“Main Valve” is defined as a pneumatic valve which allows the changing between Feed and Regeneration states of PSA/VPSA operation to occur between the two ore more beds operating in parallel. The Main Valve comprises compression valving and vacuum valving to facilitate the Feed and Regeneration states, respectively, and serves as an inlet to the air separation system.

“Compression valving” is defined as the component of the Main Valve which attaches to an external compressor line and allows for the pumping of a compressed multicomponent gas stream into the air separator system for the Feed state of PSA/VPSA operation.

“Vacuum valving” is defined as the component of the Main Valve which attaches to an external vacuum line and allows for the pumping of a vacuum into the air separator system for the Regeneration state of PSA/VPSA operation.

“EQ Valve” is defined as a pneumatic valve which ties the tail end of the two or more beds together for the Equalization state of PSA/VPSA operation to occur, and also comprises valving for collecting product gas during the Feed state of PSA/VPSA operation.

“Equalization valving” is defined as the component of the EQ Valve which connects, or blocks the connection depending on the state of the system, between the two or more beds.

“Connecting port” is defined as any space in the Main Valve or EQ Valve which provides an operable air passage between one of the sieve beds, and either the compression, vacuum, or equalization valving. Depending on the specific design of the manifold, the connecting ports may or may not be present, or may or may not be differentiable from the function-specific ports.

“Compression port” is defined as the space in the Main Valve which allows for operable air passage between the compression valving and one of the connecting ports to the adsorbent vessels, or the entrance to one of the adsorbent vessels if there is no independent connecting port.

“Vacuum port” is defined as the space in the Main Valve which allows for operable air passage between the vacuum valving and one of the connecting ports to the adsorbent vessels, or the entrance to one of the adsorbent vessels if there is no independent connecting port.

“Headspace” is defined as the total volume of space between the sorbent in the sieve bed and the valve designated for the indicated headspace—this includes any empty volume at the end of the sieve bed (between the sorbent and the manifold), any empty space within the ports to the valve, any empty space leading to the entrance of the valve or tube. Some empty space that is included in one headspace may also be considered part of another headspace, for example the inlet manifold may have some volume of space where it connects to the sorbent bed, between the sorbent bed and both the compression port and the vacuum port. This volume of space is considered part of the compression headspace, as well as part of the vacuum headspace.

“Downstream headspace” is defined as any headspace between the equalization valve and sieve bed, which is present in the equalization manifold, comprising the equalization headspace in the connecting ports.

“Upstream headspace” is defined as any headspace between the sieve bed and valves in the feed manifold, comprising the vacuum headspace and the compression headspace depending on the state of the system.

The present set of manifolds enhances the transportability, oxygen performance, and compactness of portable oxygen concentrators and other multicomponent gas stream separators using pressure-swing-adsorption, vacuum-swing-adsorption, and/or vacuum-pressure-swing-adsorption. The manifolds are composed of a Feed Manifold and a Equalization Manifold. The manifold designs eliminate the need of a purge mechanism for concentrating oxygen from any air separation design. The manifolds allow for pneumatic valving, offering significantly higher valve flow coefficients (Cv), maximizing flow throughput from any compressor and/or vacuum into the air separation device.

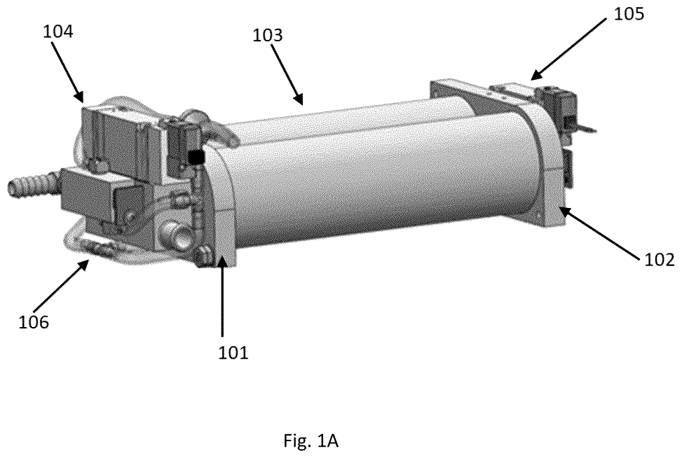

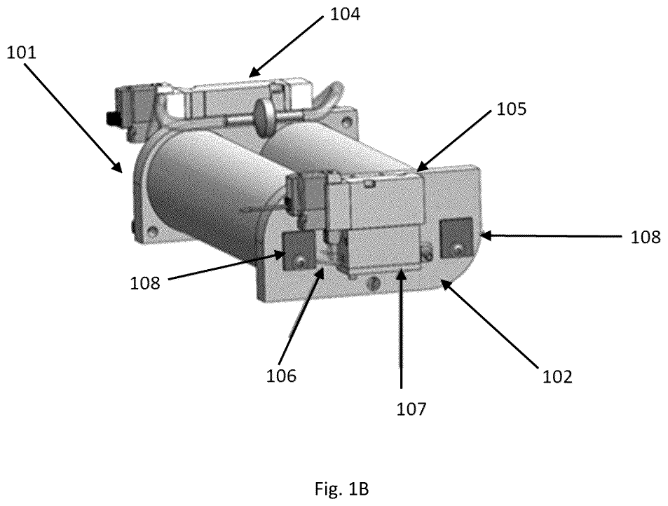

The set of manifolds consists of two primary parts, the Inlet or Feed Manifold, and the Exit or Equalization Manifold. These parts can be made from steel, aluminum, metal plating, or other additive manufacturing-based material that can withstand 50 psia and not allow diffusion of the gas across the material's medium. Together they hold two adsorbent vessels, two valves, and allow the correct pathways for the PSA/VPSA four states to enable high purity oxygen output for gas separation of air. However, no separate Purge state is required as some high purity oxygen is recycled to effectively purge the sorbent bed during the Equalization state. FIG. 1 shows an oxygen module with the two manifolds holding all the components. The manifolds have specific port designs to mount two valves. The Main Valve is a five-port-two-way valve which changes the PSA/VPSA cycle between Feed and Regeneration states. The EQ Valve is a two-port-two-way valve which ties the tail end of the beds together for Equalization of the PSA/VPSA operation. The adsorbent vessels are sealed by an O-ring which sits in both sides of the radial rod gland of the manifolds. The two manifolds and adsorbent vessels are held together by two tie rods where the Equalization Manifold has internal threads for holding in the tie rod and the Feed Manifold has through holes to allow the tie rods to pass through the manifold for a nut fitting to make the final junction.

The set of manifolds can be used on any set of at least 2 sorbent beds, contained in separate vessels which may be cylindrical with length/diameter (L/D) dimensions ranging from 1 to 5. Preferably, the entire system weighs less than 32 lbs, or less than 40 lbs, less than 35 lbs, less than 30 lbs, less than 25 lbs.

FIG. 1 shows the manifolds in use on an oxygen concentrator with two sieve beds. The Feed Manifold (101) and Equalization Manifold (102) attach to the ends of two adsorbent vessels (103) containing sorbent beds, where the Feed Manifold is attached to the feed end and the Equalization Manifold is attached to the product end of the sorbent beds. A Main Valve (104) operably attaches to the Feed Manifold, while an EQ Valve (105) operably attaches to the Equalization Manifold. Pneumatic lines (106) attach to the valves and carry compressed air. The Equalization Manifold further comprises a product port (107) containing check valves, and pressure sensors (108).

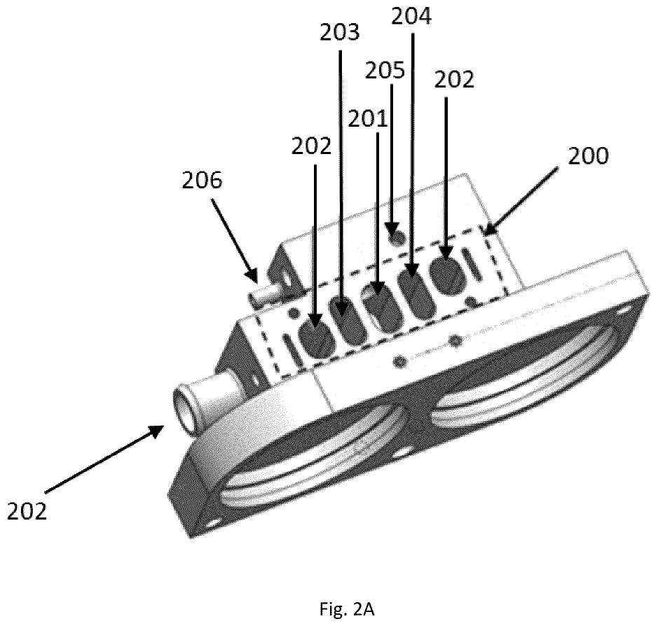

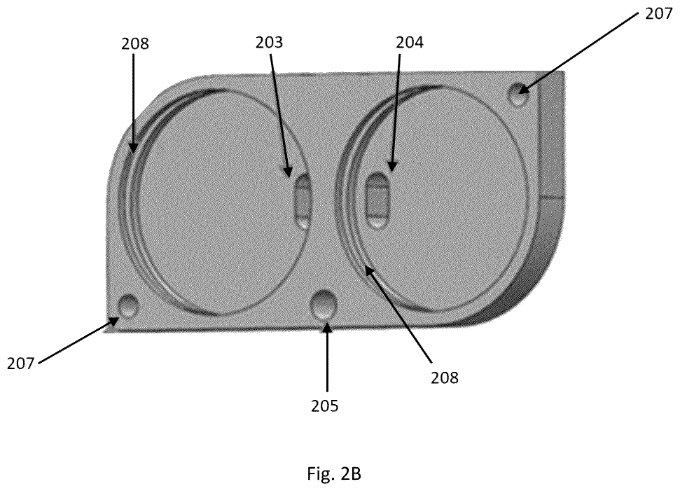

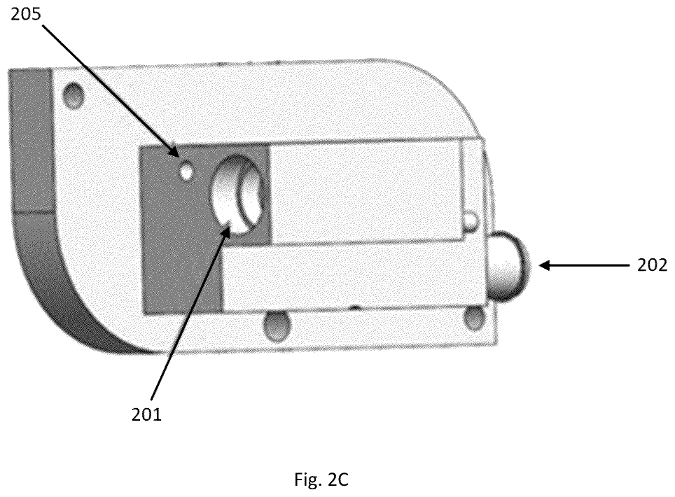

The outside of the Feed Manifold is shown in three perspectives in FIG. 2. FIG. 2A shows a top view of the Main Valve port (200) comprising a compression port (201), vacuum ports (202), a first connecting port (203) between the first sieve bed and the compression/vacuum ports, a second connecting port (204) between the second sieve bed and the compression/vacuum ports, a pneumatic port (205), and pressure sensor mount (206). FIG. 2B shows the inner side view of the manifold, where the manifold connects to the sieve beds through the first connecting port (203) and the second connecting port (204). The pneumatic port (205), tie rod mounts (207), and radial O-ring seal glands (208) promote functionality of the entire connected gas stream separation system. FIG. 2C shows the outer side view of the manifold, where the compression port (201), vacuum port (202), and pneumatic port (205) connect to external lines.

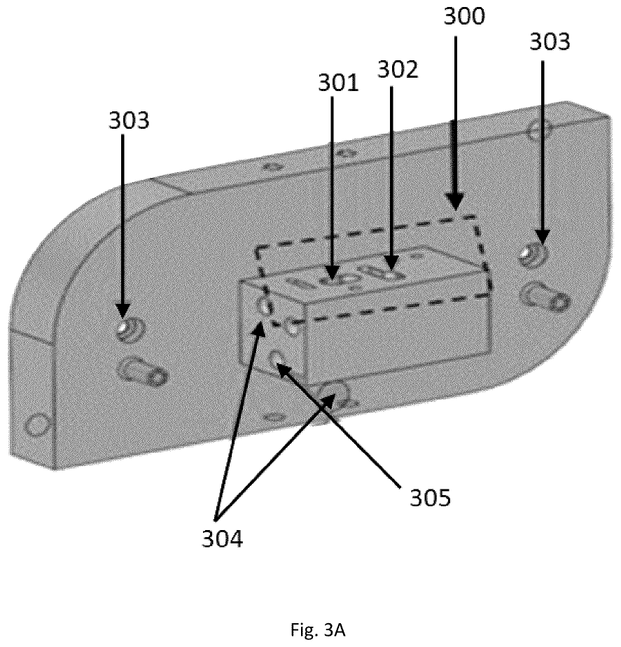

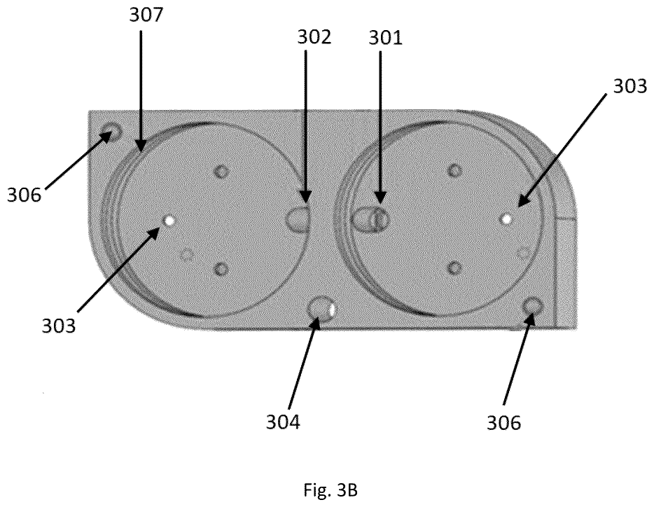

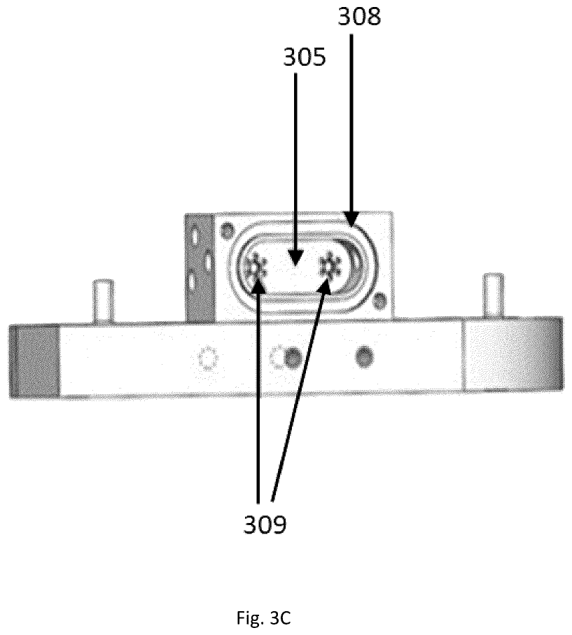

The Equalization Manifold is shown in three perspectives in FIG. 3. FIG. 3A shows the EQ Valve port (300) comprising a first connecting port (301) between the first sieve bed and the internal equalization valve, a second connecting port (302) between the second sieve bed and the internal equalization valve, pneumatic ports (304), and an O₂ product chamber (305). FIG. 3B shows the inner side view of the manifold, where the manifold connects to the sieve beds through connecting ports (301). Pressure sensor mounts (303), pneumatic port (304), tie rod mounts (306), and radial O-ring seal glands (307) promote functionality of the entire connected gas stream separation system. FIG. 3C shows the under side view of the manifold, where the O₂ product chamber (305) comprises a face seal (308) for miniature umbrella check valves (309), where there is at least one check valve on each side of the equalization valving connecting each separation vessel to the O2 chamber.

The Main Valve port comprises the compression port which connects the compression valving to the sieve beds, and vacuum ports which connect the vacuum valving to the sieve beds. The compression port(s) are also connected externally to a compression line, and the vacuum port(s) are also connected externally to a vacuum line. The adsorbent beds are connected to the compression and vacuum ports through connecting ports between a first adsorbent bed and the manifold, and a second adsorbent bed and the manifold. Each connecting port in the Feed Manifold is connected to a different sieve bed, at least one vacuum port, and at least one compression port. A pneumatic port takes a sample of the compressed air which has not passed through the adsorbent vessel to actuate the pneumatic valves required to move the valve for changing the PSA/VPSA state from Feed to Regeneration, and Regeneration to Feed in each sieve bed. The Main Valve transitions the beds from initially connecting to the compression port, to then connecting to the vacuum port, or vice versa depending on which cycle the beds are on. One bed will be in the Feed state, connecting to the compression port, while the other is in Regeneration state, connecting to the vacuum port. After saturation of the Feed state is reached, meaning the sorbent has adsorbed a maximum amount of undesired gases, the operation will switch between adsorbent vessels so that the Feed state is on an unsaturated adsorbent vessel while the saturated adsorbent vessel enters the Regeneration state. The switch for each bed occurs simultaneously within the Main Valve by the pneumatic valving moving each connecting port between being connected to the compression valving or the vacuum valving. The cycle is repeated to allow for constant gas separation for O₂ production on the exit end of the reactors.

The Equalization Manifold mates to the tail end of the adsorbent vessels and mounts and seals the EQ Valve. The EQ Valve port comprises connecting ports connecting the first adsorbent and the second adsorbent bed to the manifold. It is a two-port-two-way spool valve that allows the PSA/VPSA cycle to enter the Equalization state. The Equalization Manifold effectively purges the sorbent bed, while not requiring a separate Purge state, by directing very pure O₂ collected in the headspace of one sorbent bed over the other sorbent bed. The headspace is calculated to maintain maximal O₂ collection efficiency while providing enough O₂ to effectively purge the sorbent bed. When the valve is normally closed, the adsorbent vessels are independent of one another, and the tail end flow cannot pass between the adsorbent vessels. During this time, the adsorbent vessel which is in the Feed state can pass the high product O₂ gas through the miniature check valves and into an O₂ product chamber so that the gas may leave the system to some other subsystem to store the gas. The miniature check valves impede the high purity O₂ streamline from back-flowing into the opposing adsorbent bed in the Regeneration state.

The pneumatic line from a flexible tubing is routed from the Feed Manifold to the Equalization Manifold and ties into the manifold blocks at pneumatic ports. The pneumatic line is responsible for actuation of the spool valve to change the PSA/VPSA cycle from equalization states in the Equalization Manifold. The pneumatic line also actuates the compression valving and vacuum valving in the Feed Manifold.

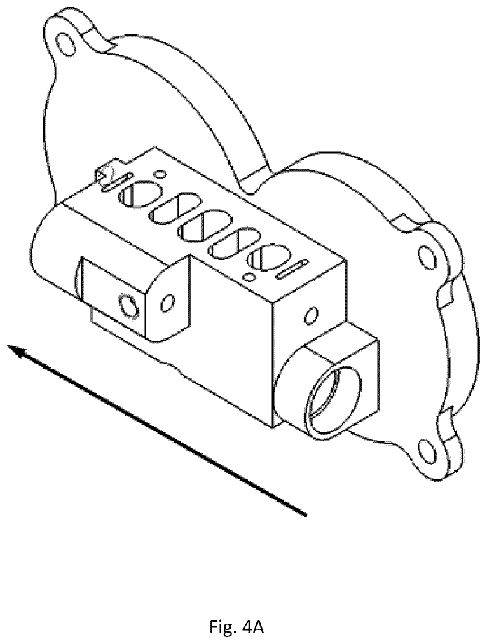

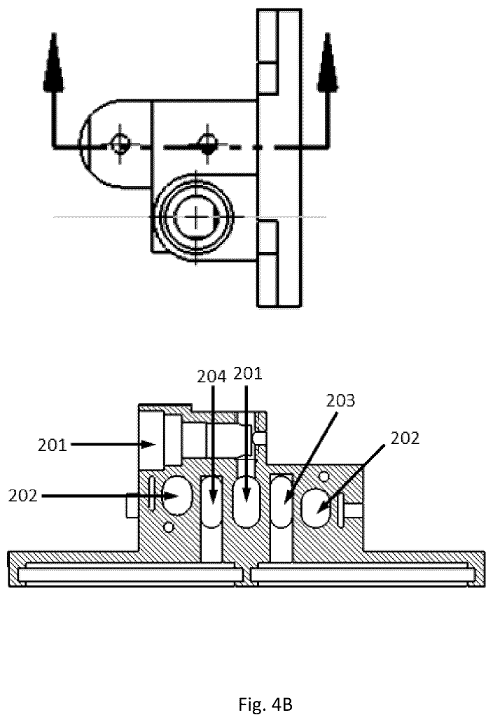

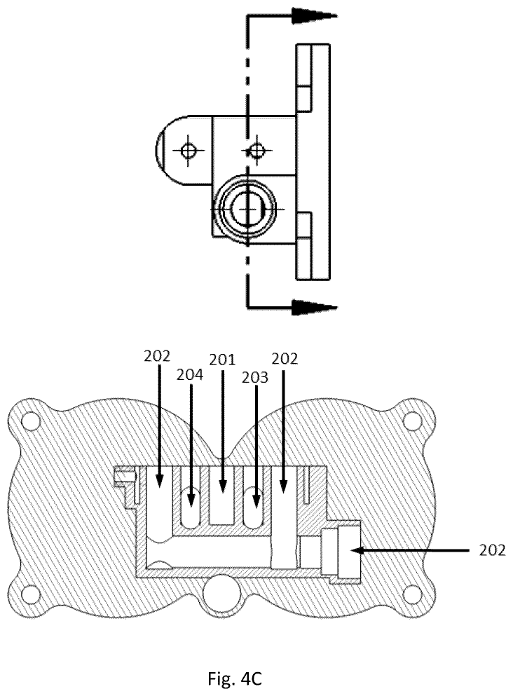

FIG. 4 shows cutout views of the Feed Manifold, clarifying spatial connections through the various ports. FIG. 4A directs the line of sight for the horizontal cutout view of FIG. 4B and the vertical cutout view of FIG. 4C. The horizontal cutout view in FIG. 4B shows the first connecting port (203) between the first sieve bed and the compression/vacuum ports, the second connecting port (204) between the second sieve bed and the compression/vacuum ports, vacuum ports (202), and compression port (201). The vertical cutout view in FIG. 4C shows another view of the first connecting port (203) between the first sieve bed and the compression/vacuum ports, a second connecting port (204) between the second sieve bed and the compression/vacuum ports, vacuum ports (202), and compression port (201). FIG. 4 shows one embodiment of how the ports may internally connect to one another. In this embodiment an external compression pump connects to the manifold on the upper side through compression port (201), and an external compression pump connects to the manifold on the lower side through vacuum port (202).

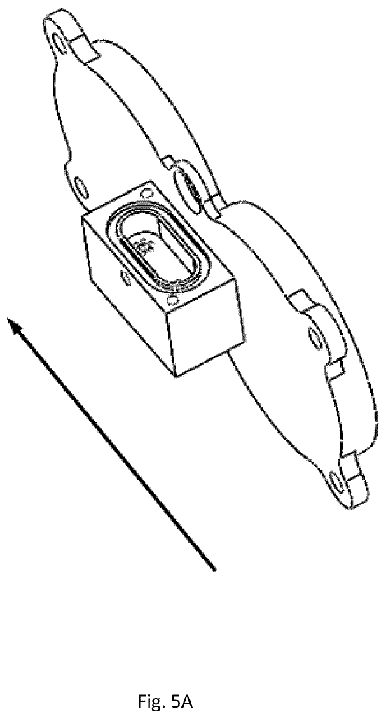

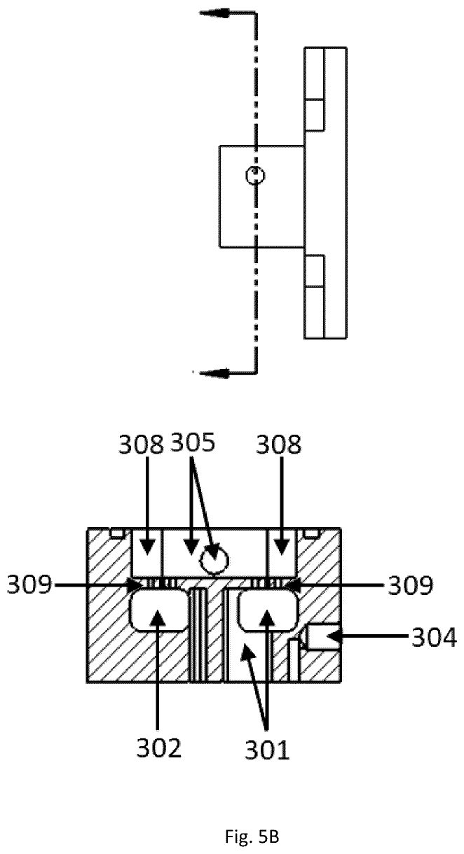

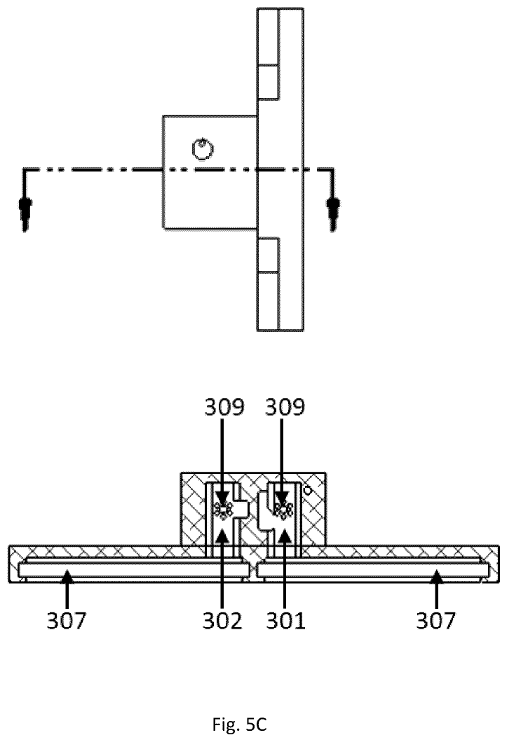

FIG. 5 shows cutout views of the Equalization Manifold, clarifying spatial connections through the various ports. FIG. 5A directs the light of sight for the vertical cutout view of FIG. 5B and the horizontal cutout view of FIG. 5C. The vertical cutout view in FIG. 5B shows the first connecting port (301) between the first sieve bed and the internal equalization valve, the second connecting port (302) between the second sieve bed and the internal equalization valve, pneumatic port (304), and O₂ product chamber (305) comprising a face seal (308) and miniature umbrella check valves (309). FIG. 5C shows another view of the first connecting port (301) between the first sieve bed and the internal equalization valve, the second connecting port (302) between the second sieve bed and the internal equalization valve, and the miniature umbrella check valves (309), as well as the outside of the radial O-ring seal glands (307).

A critical embodiment of the set of manifolds is the ratio of upstream headspace to the volume of sorbent in the combined sieve beds. Alternatively, the ratio of upstream headspace to the mass of sorbent in the combined sieve beds. Upstream headspace is the volume of empty space between the main valving and the sieve bed, which is comprised of both vacuum headspace and compression headspace. The vacuum headspace is equal to the total space in a vacuum port and whichever connecting port is connecting the vacuum port to an adsorbent bed in the Regeneration state, depending on the state of the system. The compression headspace is equal to the total space in a compression port and whichever connecting port is connecting the compression port to an adsorbent bed in the Feed state, depending on the state of the system.

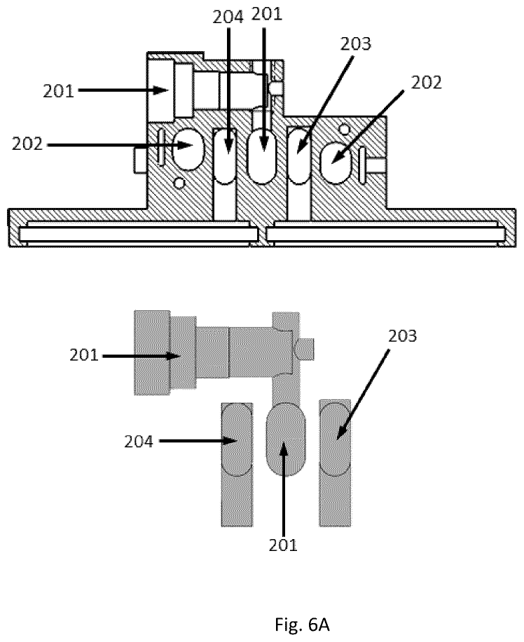

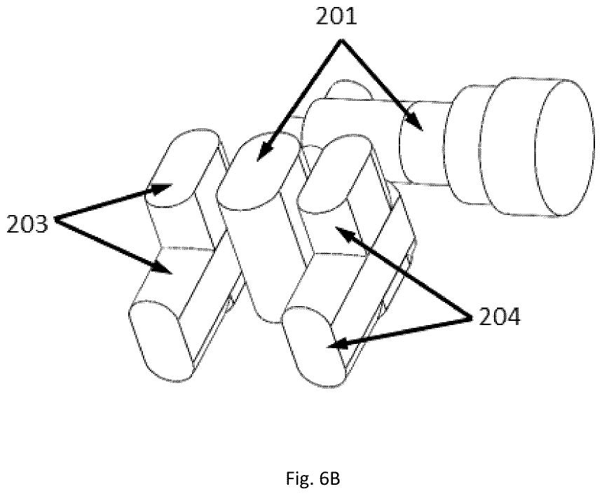

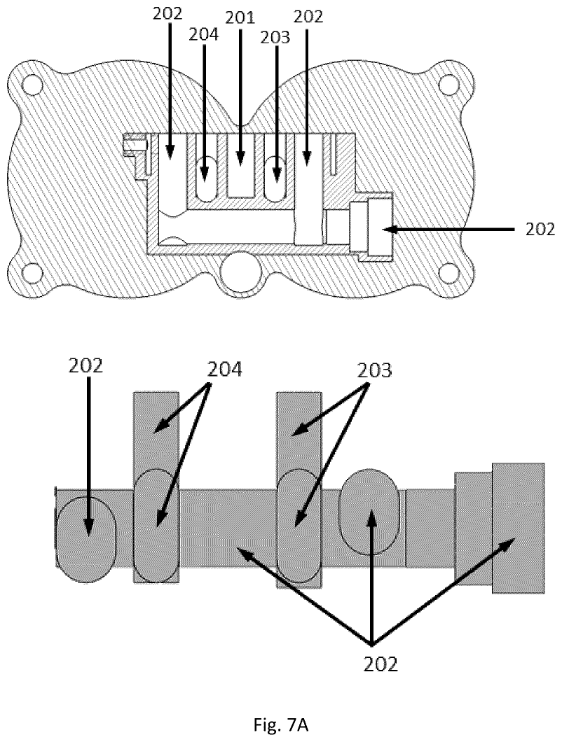

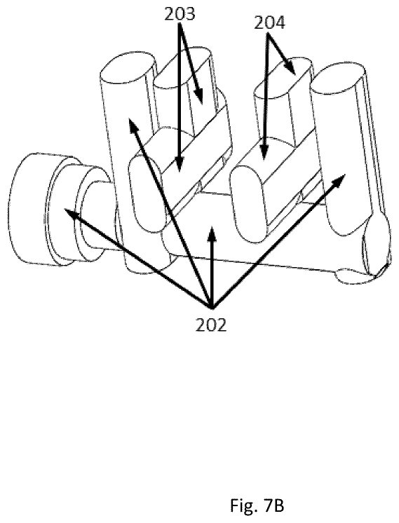

FIG. 6 shows the ports which could comprise compression headspace. FIG. 6A is a 2-dimensional depiction (horizontal slice, view from underneath) of possible compression headspace, comprising the first connecting port (203) between the first sieve bed and the compression/vacuum ports, the second connecting port (204) between the second sieve bed and the compression/vacuum ports, and the compression port (201). The 2-dimensional slice also shows the vacuum ports (202). FIG. 6B is a 3-dimensional depiction of the negative space in the manifold that is possibly compression headspace, comprising the first connecting port (203) between the first sieve bed and the compression/vacuum ports, the second connecting port (204) between the second sieve bed and the compression/vacuum ports, and the compression port (201). FIG. 7 shows the ports which could comprise vacuum headspace. FIG. 7A is a 2-dimensional depiction (vertical slice, cutout shows view from above) of possible vacuum headspace, comprising the first connecting port (203) between the first sieve bed and the compression/vacuum ports, the second connecting port (204) between the second sieve bed and the compression/vacuum ports, and the vacuum ports (202). The 2-dimensional slice also shows the compression port (201). FIG. 7B is a 3-dimensional depiction of the negative space in the manifold that is possibly vacuum headspace, comprising the first connecting port (203) between the first sieve bed and the compression/vacuum ports, the second connecting port (204) between the second sieve bed and the compression/vacuum ports, and the vacuum ports (202).

The total upstream headspace (vacuum headspace+compression headspace) may be 2-6% of the total volume of all sieve material used in all combined sieve beds, more preferably 3-5%, most preferably 4-5% or about 4.5% or 4.25-4.75%. Preferably, the vacuum headspace is 0.5-2.5%, more preferably 1-2%, most preferably about 1.4% or 1.2-1.6%. Preferably, the compression headspace is 0.5-2.5%, more preferably 0.5-1.5%, most preferably about 0.6% or 0.55-0.7%.

Another critical embodiment of the set of manifolds is the ratio of downstream headspace to the volume of sorbent in the combined sieve beds. Downstream headspace is the volume of any empty space between the EQ valve and the sieve bed, which is equal to the space in the connecting ports. This headspace is referred to as the downstream headspace for equalization.

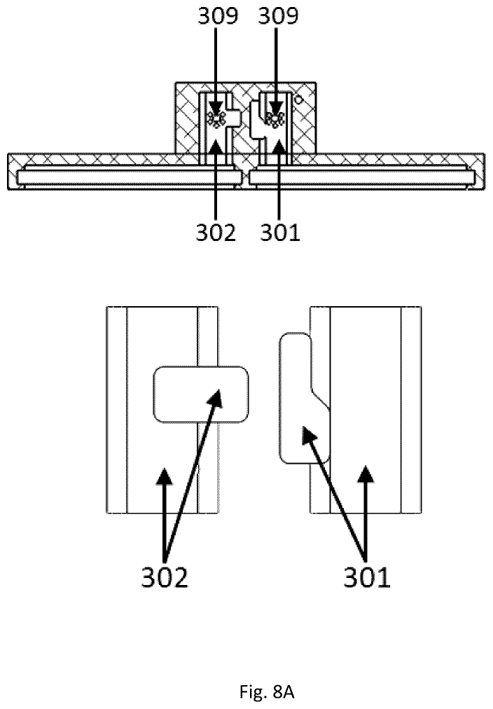

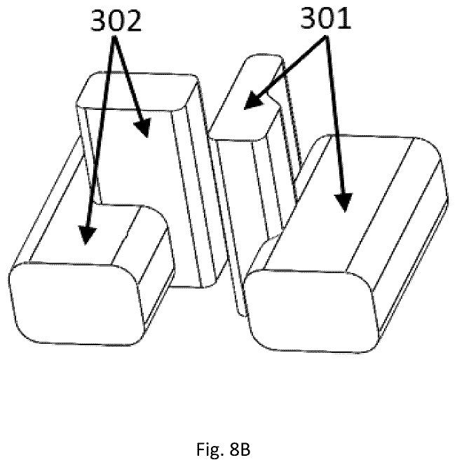

FIG. 8 shows the ports which could comprise equalization headspace. FIG. 8A is a 2-dimensional depiction of possible equalization headspace, comprising the first connecting port (301) between the first sieve bed and the internal equalization valve, the second connecting port (302) between the second sieve bed and the internal equalization valve. The 2-dimensional slice also shows the miniature umbrella valves (309). FIG. 8B is a 3-dimensional depiction of the negative space in the manifold that is possibly compression headspace, comprising the first connecting port (301) between the first sieve bed and the internal equalization valve, the second connecting port (302) between the second sieve bed and the internal equalization valve.

The total Downstream headspace for equalization may be 2-6% of the total volume of all sieve material used in all combined sieve beds, more preferably 3-5%, most preferably 4-5% or about 4.5%.

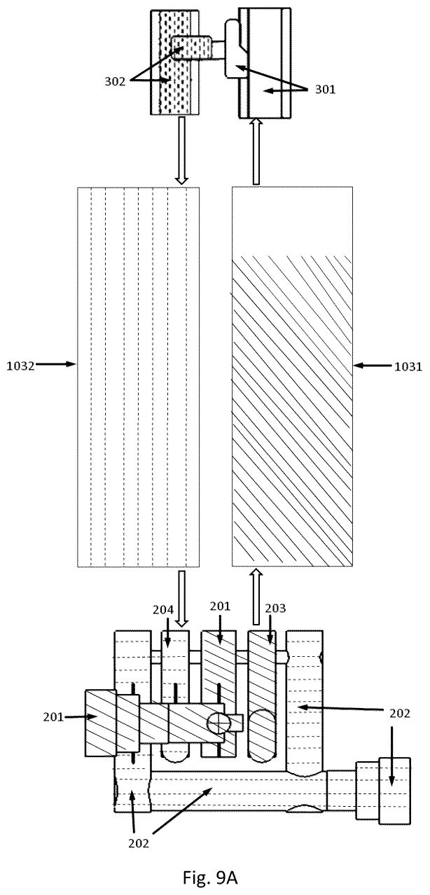

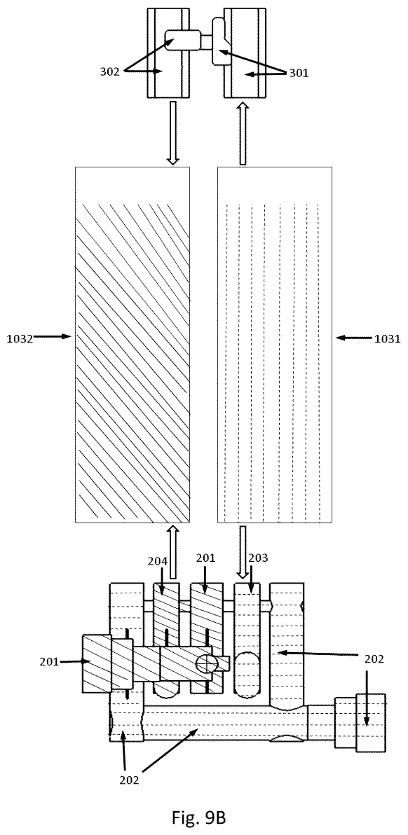

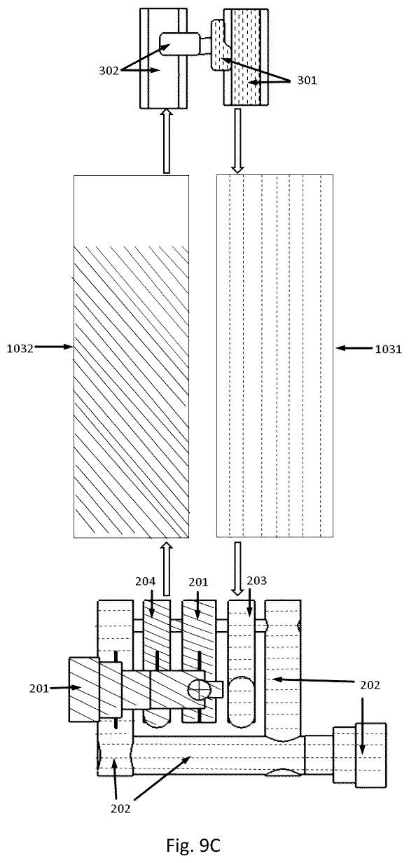

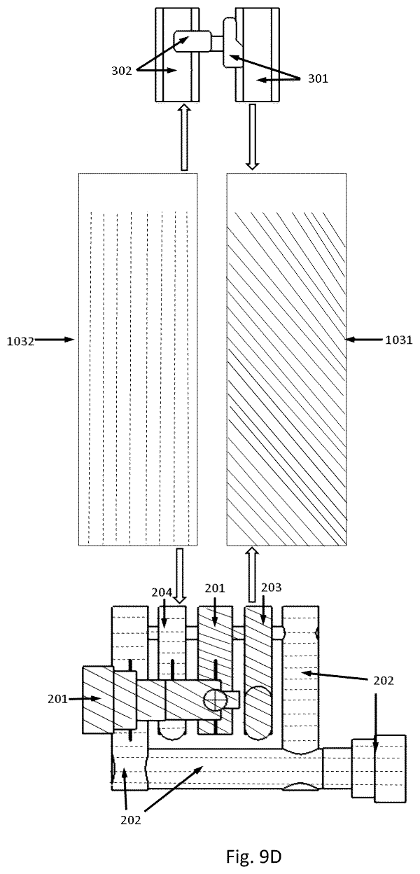

FIG. 9 shows the components which make up compression, vacuum, and equalization headspaces through a single operation cycle, while connected to a first sieve bed (1031) and a second sieve bed (1032). The Feed Manifold is shown from a front-view (the Main Valve attaches above the figure, and the sieve beds attach behind the figure; same view as FIG. 7A). The total upstream headspace is the combined volume of all the ports in the Feed Manifold, comprising the compression port (201), the vacuum port (202), the first connecting port (203), and the second connecting port (204). The Equalization Manifold is shown from a top-view (the EQ Valve attaches in front of the figure, and the sieve beds attach below the figure; same view as FIG. 8A). The total downstream headspace is the combined volume of all the ports in the Equalization Manifold, comprising the first connecting port (301) and the second connecting port (302). For both manifolds and sieve beds, solid lines designate ports and beds in the compression/feed state; dashed lines designate ports and beds in the vacuum/regeneration state; white space in the ports and beds represent purified O₂ (i.e., UHP grade O₂).

FIG. 9A shows an example first state, where the first sieve bed (1031) is in the compression/feed state, and the second sieve bed (1032) is in the vacuum state. The Main Valve (not pictured) connects the first connecting port to the compression port, and the second connecting port to the vacuum port. The compression headspace is the combined volume in the first connecting port (203) and in the compression port (201). The vacuum headspace is the combined volume in the second connecting port (204) and the vacuum port (202). The EQ valve (not pictured) keeps the first connecting port (301) and the second connecting port (302) on the Equalization Manifold separate from one another. A compressed air stream is concentrated as it flows through the first sieve bed (1031) into the first connecting port, which may ultimately lead to a collection vessel (not pictured). Some purified O₂ collects in the headspace in the first connecting port (301).

FIG. 9B shows an example second state, where the first sieve bed (1031) and the second sieve bed (1032) are both in the equalization state. The equalization state begins simultaneously with the switching of the ports on the Feed Manifold, so the second sieve bed, which was previously in the vacuum state, has O₂ entering from downstream and compressed air coming in from upstream. The first sieve bed begins the vacuum/regeneration state. The EQ valve (not pictured) connects the first connecting port (301) and the second connecting port (302) on the Equalization Manifold to one another. The combined volume of the first connecting port and the second connecting port on the Equalization Manifold is the downstream headspace. The small amount of purified oxygen collected in the first connecting port (301) during the purification state shown in FIG. 9A flows through the second connecting port (302) and into the second sieve bed (1032) to effectively purify the sorbent in the sieve bed prior to entering the compression state. The Main Valve (not pictured) connects the first connecting port (203) to the vacuum port (202), and their combined volume is the vacuum headspace. The second connecting port (204) connects to the compression port (201), and their combined volume is the compression headspace.

FIG. 9C shows an example third state, where the first sieve bed (1031) is in the vacuum/regeneration state, and the second sieve bed (1032) is in the compression/feed state. The Main Valve (not pictured) connects the first connecting port to the vacuum port, and the second connecting port to the compression port. The compression headspace is the combined volume in the second connecting port (204) and in the compression port (201). The vacuum headspace is the combined volume in the first connecting port (203) and the vacuum port (202). The EQ valve (not pictured) keeps the first connecting port (301) and the second connecting port (302) on the Equalization Manifold separate from one another. A compressed air stream is concentrated as it flows through the second sieve bed (1032) into the second connecting port, which may ultimately lead to a collection vessel (not pictured). Some purified O₂ collects in the headspace in the second connecting port (302).

FIG. 9D shows an example fourth state, where the first sieve bed (1031) and the second sieve bed (1032) are both in the equalization state. The equalization state begins simultaneously with the switching of the ports on the Feed Manifold, so the first sieve bed, which was previously in the vacuum state, has O₂ entering from downstream and compressed air coming in from upstream. The EQ valve (not pictured) connects the first connecting port (301) and the second connecting port (302) on the Equalization Manifold to one another. The combined volume of the first connecting port and the second connecting port on the Equalization Manifold is the downstream headspace. The small amount of purified oxygen collected in the second connecting port (302) during the purification state shown in FIG. 9C flows through the first connecting port (301) and into the first sieve bed (1031) to effectively purify the sorbent in the sieve bed prior to entering the compression state. The Main Valve (not pictured) connects the first connecting port (203) to the compression port (201), and their combined volume is the compression headspace. The second connecting port (204) connects to the vacuum port (202), and their combined volume is the vacuum headspace. After this example fourth state, the oxygen concentrator may continue the cycle from the state shown in FIG. 9A.

As shown in the example cycle in FIG. 9, the connecting ports on the Feed Manifold can be individually counted as the compression headspace or the vacuum headspace, depending on the state of the system. They remain connected to opposite ports (vacuum port versus compression port) during oxygen concentration, except during the equalization state where each they are connected to neither.

Also critical is the ratio of valve flow coefficient to the total sorbent volume of the combined sieve beds (Cv/mL). The compression valving, vacuum valving, and equalization valving all have an inherent flow coefficient (Cv). The compression valving flow coefficient, vacuum valving flow coefficient, and equalization valving flow coefficient (Cv) to sorbent volume ratios may be

$$0.0005 - 0.005 ( \frac{gal}{min} ) ( \frac{1}{{L}_{S}} ) .$$

with gal/min at 1 psi being standard Cv units, and Lₛ being total liters of sorbent in all sieve beds in the entire system. More preferably, the ratio each Cv to total sorbent volume is

$$0.001 - 0.003 ( \frac{gal}{min} ) ( \frac{1}{{L}_{S}} ) ,$$

and most preferably the ratios for compression Cv, vacuum Cv, and equalization Cv are 0.0017, 0.0017, and

$$0.0013 ( \frac{gal}{min} ) ( \frac{1}{{L}_{S}} )$$

respectfully. The most preferable ratios may be within a reasonable range for error, being 0.0017±0.00017, 0.0017±0.00017, and 0.0013±0.00013 respectfully.

The manifolds are used for purifying oxygen or concentrating oxygen for medical purposes using any air separation sorbent to provide a sufficient volume of sweep gas consisting of high purities of a least-preferential-able composition which improves the removal of a preferential gas that has been adsorbed by the sieve beds during the regeneration state using an equalization state. They also may purify a mixed gas stream, or concentrate a specific gas composition through the use of any pressure-swing-adsorption or vacuum-pressure-swing-adsorption without the use of a purge mechanism. They can be scaled in size to accommodate a variety of air separation outputs ranging from 2-30 LPM. Another optional method can scale the manifolds while accommodating the ratios by scaling the total volume of adsorbent from 100-6000 mL.

In an embodiment, the adsorption pressure is 1.2-10 bar, which provides the sufficient volume of oxygen sweep gas during the equalization state. The desorption pressure is 0.03-1.1 bar during the regeneration state. These pressure specifications allow the present manifolds to extend to either a PSA or VPSA system.

For the manifolds to properly work, at least two sieve beds must be used in parallel in the PSA/VPSA system. The manifolds can be extended to more than two sieve beds by maintaining the ratios described herein. There can be any odd number or any even number of sieve beds added. Each sieve bed may include plural or singular adsorbent elements. Additionally, any number of manifolds can be used so long as the overall ratios of combined headspaces are preserved. When more than two manifolds are used in a system, at least one Feed Manifold and at least one Equalization Manifold must be present. Alternative designs of manifolds can be used (i.e., more or fewer compression, vacuum, or pneumatic ports or valves) so long as the overall ratios of combined headspaces are preserved.

In an expanded system, each headspace that does equalization, vacuum, or compression will be totaled and ratioed against the total number of sieve beds. For example, all the manifolds that allow multiple sieve beds to compress will each individually be summed for the total headspace volume for compression. The same will be done to determine a total headspace volume for vacuum. The two headspaces will be summed to determine a total upstream headspace, which will be divided by the total volume of sorbent within all the multiple sieve beds combined. The calculation is done based on the total sieve material used, not the number of beds, so the total headspace for equalization, vacuum, or compression will be divided by the total amount of sieve material used in the entire system.

While the manifolds are described and illustrated with respect to an adsorption system for separating and concentrating oxygen, it should be noted that the manifolds may be used on any separation system with a plurality of adsorbent beds for recovering valuable gaseous products from a multicomponent gas stream, such as hydrogen, carbon oxides, synthesis gas, light hydrocarbons, and the like. The separation system must employ either a pressure-swing-adsorption technology or a vacuum-pressure-swing-adsorption technology, either of which may use the assistance of temperature swing for adsorption.

The valving within the manifolds, and their residence ports and/or connecting ports may be varied in design, and many valving technologies may be used, so long as the functionality and critical headspaces, flow rates, and adsorption pressures are maintained.

Example 1: a two sieve bed which uses any combination of valving while maintaining the necessary pressures, headspaces, and Cv to concentrate oxygen.

Example 2: a (3+2n) number of sieve beds which uses any combination of valving while maintaining the necessary pressures, headspaces, and Cv to concentrate oxygen.

Example 3: a (2+2n) number of sieve beds where two sieve beds are always in parallel operation which uses any combination of valving while maintaining the necessary pressures, headspaces, and Cv to concentrate oxygen.

1. A pressure-swing-adsorption (PSA) or vacuum-pressure-swing-adsorption (VPSA) oxygen concentrator device comprising:
a) at least 2 sorbent beds, each having a feed end and a product end;
b) at least one feed manifold attached to the feed end of the at least 2 sorbent beds; and,
c) at least one equalization manifold attached to the product end of the at least 2 sorbent beds;
wherein, the oxygen concentrator device does not comprise a purge mechanism;
wherein, the oxygen concentrator device weighs less than 32 pounds; and,
wherein, the oxygen concentrator device produces a concentrated gas stream of at least 70-95% oxygen.

2. A pressure-swing-adsorption (PSA) or vacuum-pressure-swing-adsorption (VPSA) oxygen concentrator device comprising:
a) at least 2 sorbent beds, each having a feed end, a product end, and a total sorbent volume within the combined sorbent beds;
b) at least one feed manifold attached to the feed end of the at least 2 sorbent beds; and,
c) at least one equalization manifold attached to the product end of the at least 2 sorbent beds;
wherein,
the at least one feed manifold further comprises:
at least one compression port connecting the at least 2 sorbent beds to a compression valve, wherein the compression valve has a compression valve flow coefficient (compression Cv);
at least one vacuum port connecting the at least 2 sorbent beds to a vacuum valve, wherein the vacuum valve has a vacuum valve flow coefficient (vacuum Cv); and,
an upstream headspace, comprising a compression headspace between the sorbent beds and the compression valve, and a vacuum headspace between the sorbent beds and the vacuum valve;
the at least one equalization manifold further comprises:
at least one equalization port connecting the at least 2 sorbent beds to an equalization valve, wherein the equalization valve has an equalization valve flow coefficient (equalization Cv); and,
a downstream headspace, comprising an equalization headspace between the sorbent beds and the equalization valve;
a ratio of the compression Cv to the total sorbent volume is
$$0.0005 - 0.005 ( \frac{gal}{min} ) ( \frac{1}{{L}_{S}} ) ;$$
a ratio of the vacuum Cv to the total sorbent volume is
$$0.0005 - 0.005 ( \frac{gal}{min} ) ( \frac{1}{{L}_{S}} ) ;$$
a ratio of the equalization Cv to the total sorbent volume is
$$0.0005 - 0.005 ( \frac{gal}{min} ) ( \frac{1}{{L}_{S}} ) ;$$
the upstream headspace is 2-6% of the total sorbent volume; and,
the downstream headspace is 2-6% of the total sorbent volume.

3. The oxygen concentrator device as in claim 2, wherein the upstream headspace is 3-5% of the total sorbent volume.

4. The oxygen concentrator device as in claim 2, wherein the vacuum headspace is 0.5-2.5% of the total sorbent volume and the compression headspace is 0.5-2.5% of the total sorbent volume.

5. The oxygen concentrator device as in claim 2, wherein the downstream headspace is 3-5% of the total sorbent volume.

6. The oxygen concentrator device as in claim 2, wherein the feed manifold comprises a five-port-two-way valve and the equalization manifold comprises a two-port-two-way valve.

7. The oxygen concentrator device as in claim 2, wherein the ratio of the compression Cv to the total sorbent volume, the ratio of the vacuum Cv to the total sorbent volume, and the ratio of the equalization Cv to the total sorbent volume are each
$$0.0001 - 0.003 ( \frac{gal}{min} ) ( \frac{1}{{L}_{S}} ) .$$

8. The oxygen concentrator device as in claim 2, wherein the total sorbent volume is 100-6000 mL.

9. The oxygen concentrator device as in claim 2, wherein the at least 2 sorbent beds comprise at least 3 sorbent beds, and wherein at least 2 sorbent beds operate in parallel.

10. The oxygen concentrator device as in claim 2, wherein the oxygen concentrator device further comprises a temperature-swing-adsorption device.

11. The oxygen concentrator device as in claim 2, wherein the oxygen concentrator device does not comprise a purge mechanism.

12. The oxygen concentrator device as in claim 2, wherein the oxygen concentrator device weighs less than 32 lbs.

13. The oxygen concentrator device as in claim 2, wherein the at least one feed manifold further comprises a pneumatic port connecting to at least one pressure line for actuation of the compression valve and the vacuum valve.

14. The oxygen concentrator device as in claim 2, wherein the equalization valve further comprises at least one check valve between each of the at least 2 sorbent beds and the equalization valve, wherein the check valve connects each of the at least 2 sorbent beds to an O₂ product chamber.

15. A method of concentrating oxygen for medical purposes, the steps comprising:
a) providing a pressure-swing-adsorption (PSA) or vacuum-pressure-swing-adsorption (VPSA) oxygen concentrator device comprising at least 2 sorbent beds having a feed end and a product end, and a total sorbent volume within the combined sorbent beds;
b) attaching at least one feed manifold to the feed end of the at least 2 sorbent beds, wherein the at least one feed manifold comprises:
at least one compression port connecting the at least 2 sorbent beds to a compression valve;
at least one vacuum port connecting the at least 2 sorbent beds to a vacuum valve; and,
an upstream headspace;
c) attaching at least one equalization manifold to the product end of the at least 2 sorbent beds, wherein the at least one equalization manifold comprises:
at least one equalization port connecting the at least 2 sorbent beds to an equalization valve; and,
a downstream headspace;
d) operating the PSA or VPSA oxygen concentrator device, wherein operating the PSA or VPSA oxygen concentrator device does not comprise separately purging the sieve bed with an unused gas; and,
e) producing a concentrated gas stream comprising 50-90% oxygen.

16. The method for concentrating oxygen for medical purposes as in claim 15, wherein the upstream headspace is 2-6% of the total sorbent volume.

17. The method for concentrating oxygen for medical purposes as in claim 15, wherein the downstream headspace is 2-6% of the total sorbent volume.

18. The method for concentrating oxygen for medical purposes as in claim 15, wherein the compression valve has a compression valve flow coefficient (compression Cv), and a ratio of the compression Cv to the total sorbent volume is
$$0.0005 - 0.005 ( \frac{gal}{min} ) ( \frac{1}{{L}_{S}} ) ;$$
wherein, the vacuum valve has a vacuum valve flow coefficient (vacuum Cv), and a ratio of the vacuum Cv to the total sorbent volume is
$$0.0005 - 0.005 ( \frac{gal}{min} ) ( \frac{1}{{L}_{S}} ) ;$$
and, wherein, the equalization valve has an equalization valve flow coefficient (equalization Cv), and a ratio of the equalization Cv to the total sorbent volume is
$$0.0005 - 0.005 ( \frac{gal}{min} ) ( \frac{1}{{L}_{S}} ) .$$

19. The method for concentrating oxygen for medical purposes as in claim 15, wherein the feed manifold further comprises a pneumatic port connecting to at least one pressure line for actuation of the compression valve and the vacuum valve.

20. The method for concentrating oxygen for medical purposes as in claim 15, wherein the concentrated gas stream comprises at least 70-95% oxygen.

21. The method for concentrating oxygen for medical purposes as in claim 20, wherein the concentrated gas stream comprises at least 80-95% oxygen.

22. The method for concentrating oxygen for medical purposes as in claim 20, wherein the equalization manifold further comprises at least one check valve between each of the at least 2 sorbent beds and the equalization valve, and wherein the check valve connects each of the at least 2 sorbent beds to an O₂ product chamber.
